(19) United States
(12) Patent Application Publication
(10) Pub. No.: US 2024/0138870 A1
(43) Pub. Date: May 2, 2024

(54) GRASPING FORCEPS

(71) Applicant: OSAKA UNIVERSITY, Osaka (JP)

(72) Inventors: Ken NAKATA, Osaka (JP); Takashi KANAMOTO, Osaka (JP)

(73) Assignee: OSAKA UNIVERSITY, Osaka (JP)

(21) Appl. No.: 18/279,464

(22) PCT Filed: Dec. 24, 2021
(86) PCT No.: PCT/JP2021/048101

§ 371 Date: Aug. 30, 2023

(30) Foreign Application Priority Data
Mar. 3, 2021 (JP) 2021-033461

(51) Int. Cl. A61B 17/29

(52) U.S. Cl. CPC A61B 17/29; A61B 2017/2901; A61B 2017/2927; A61B 2017/2939

(57) ABSTRACT

Grasping forceps include a cylindrical sheath, a shaft furnished inside the sheath, an operation unit provided near a base end of the sheath and sliding the shaft by converting rotating operation force into an axial direction of the shaft through a pin, and a grasping unit provided near a front end of the sheath and enabling upper and lower jaw units to be open and closed. The grasping unit includes a link unit that couples the upper and lower jaw units, and the front end of the shaft through a pin and opens and closes the upper and lower jaw units according to a slide of the shaft, and the link unit, when the upper and lower jaw units are in a closed state, sets facing grasping surfaces to be parallel and have a predetermined gap dimension.

### TECHNICAL FIELD

The present invention relates to grasping forceps that grasp tissue, a medical device or a product and material derived from a biologically derived material, in arthroscopic vision or direct vision treatment.

### BACKGROUND ART

Currently, in knee endoscopic surgery by use of an arthroscope, specialized forceps capable of suitably grasping without damaging or destroying tissue, a medical device or a product and material derived from a biologically derived material, are unknown. Conventionally, grasping forceps have been diverted by forceps to be used in arthrotomy or excision forceps for an endoscope for another purpose. Therefore, accurate grasping operation without damaging, destroying, or dropping the tissue has had certain limitations.

Patent Literature 1 describes forceps such that a rod may slide in an axial direction when a handle is operated so as to be tightly grasped and move a pair of grasping members at a front end unit from an open state to a closed state through a link mechanism. These forceps, when being shifted to the closed state, are configured so that both grasping surfaces of the pair of grasping members may keep a predetermined gap and become parallel, which makes it possible to grasp tissue or the like, in that state.

### CITATION LIST

### Patent Literature

Patent Literature 1: WO2011/111271

### SUMMARY OF INVENTION

### Technical Problem

However, the forceps described in Patent Literature 1 are structured so that, when the handle is further tightly grasped, both the grasping surfaces of the pair of grasping members may go beyond a state of being parallel while keeping a predetermined gap and eventually close until the grasping surfaces come into contact with each other. Accordingly, in some cases, the handle may be operated to be further tightened in an attempt to further reliably grasp tissue or the like from a state in which both the grasping surfaces are in contact with each other, and, in such cases, in addition to causing damage to the tissue or the like, a part of a minute member that configures the link mechanism near the front end of the forceps may be broken under excessive load, or destroyed and dropped.

In view of the above, the present invention aims to provide grasping forceps that enable proper grasping of tissue, a medical device, or a material and product for regenerative medicine or the like.

In addition, the present invention aims to provide grasping forceps that regulate transmission of force of predetermined tightening force or more to a shaft so as to prevent inadvertent damage to the grasped tissue or the like.

### Solution to Problem

The grasping forceps according to the present invention include a tubular sheath, a shaft furnished inside the sheath, an operation unit provided near a base end of the sheath and sliding the shaft by converting rotating operation force to a handle into an axial direction of the shaft through a first pin, and a grasping unit provided near a front end of the sheath and enabling upper and lower jaw units to be open and closed, and the grasping unit includes a link unit that couples the upper and lower jaw units and the front end of the shaft through a second pin and opens and closes the upper and lower jaw units according to a slide of the shaft, and the link unit, when the upper and lower jaw units are in a closed state, sets facing grasping surfaces to be parallel and have a predetermined gap.

According to the present invention, when the upper and lower jaw units are in the closed state, the link unit is configured so that the facing grasping surfaces may be parallel and have a predetermined gap, so that various objects to be grasped are able to be accurately, reliably, and stably grasped without being damaged, broken, or dropped.

In addition, the operation unit includes a regulation unit that, in a case in which the upper and lower jaw units are in the closed state and further the rotating operation force exceeds a predetermined force, regulates transmission of the rotating operation force to the shaft. According to this configuration, even when a handle unit is overtightened, the transmission to the shaft is regulated, which prevents a structural component being an insertion unit into a body from being damaged, destroyed, and consequently dropped.

### Advantageous Effects of Invention

According to the present invention, tissue, a medical device, or a material and product for regenerative medicine or the like is able to be accurately, reliably, and stably grasped without being damaged, broken, or dropped.

In addition, according to the present invention, without inadvertently damaging to the grasped tissue or the like, by further regulating the transmission of force of a predetermined tightening force or more to a shaft, a structure component being an insertion side to a body in use is prevented from being damaged, destroyed, and consequently dropped.

### BRIEF DESCRIPTION OF DRAWINGS

FIG. 1 is an entire schematic view showing an embodiment of grasping forceps according to the present invention.

FIG. 2 is an assembly exploded view near an operation unit of the grasping forceps according to the embodiment of the present invention.

FIG. 3A and FIG. 3B are side cross-sectional views illustrating a motion of the operation unit of the grasping forceps according to the embodiment of the present invention, FIG. 3A shows an open state, and FIG. 3B shows a closed state.

FIG. 4A to FIG. 4D are views illustrating the motion of a grasping unit of the grasping forceps according to the embodiment of the present invention, FIG. 4A is a side cross-sectional view in the open state, FIG. 4B is a side cross-sectional view in the closed state, FIG. 4C is a plan view of the grasping unit, and FIG. 4D is a top view showing an example of machining of a grasping surface of a lower jaw unit.

FIG. 5A to FIG. 5D are views showing various shapes of the grasping unit, FIG. 5A is a plan view showing a shape in which upper and lower jaw units extend straight, according to the present embodiment, FIG. 5B is a plan view showing a shape in which the upper and lower jaw units bend to one of the left and right sides, for example, to the right, FIG. 5C is a side cross-sectional view showing a shape in which the upper and lower jaw units bend to one of the upper and lower directions, for example, to the upper side, and FIG. 5D is a side cross-sectional view showing a shape in which a stopper is disposed perpendicularly to one of the facing surfaces of the upper and lower jaw units, for example, the grasping surface of the lower jaw unit.

### DESCRIPTION OF EMBODIMENTS

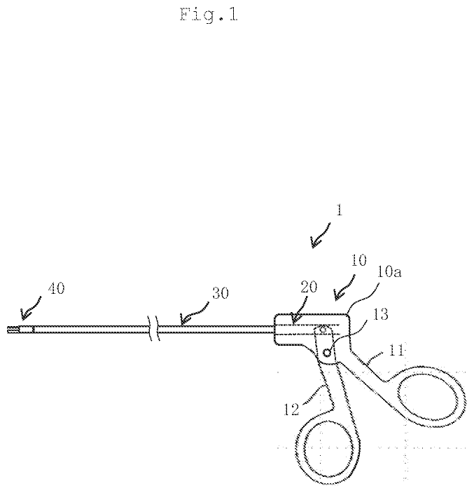

FIG. 1 is an entire schematic view showing an embodiment of grasping forceps 1 according to the present invention. The grasping forceps 1 include an operation unit 10 being a base unit, a long sheath 30 being an insertion unit to be inserted into a body in use, and a grasping unit 40 attached to a front end of the sheath 30. The components configuring each unit are basically composed of metal such as stainless steel.

The operation unit 10 includes a pair of handle units 11 and 12. The pair of handle units 11 and 12 are axially supported by a pin 13 in the middle of both arm portions and are relatively rotatable around the pin 13. The operation unit 10 has a housing unit 10a near the upper part of the handle units 11 and 12, and internally has a space (outlined by dashed lines) in a front-rear direction. The space in this front-rear direction internally includes a transmission mechanism unit 20 and enables a slide in the front-rear direction.

In the present embodiment, the handle unit 11 on the rear side is integrated with the housing unit 10a of the operation unit 10. On the other hand, the handle unit 12 on the front side is rotatable around the pin 13, and the rotating operation force to the handle unit 12, is converted into sliding power in the front-rear direction at the transmission mechanism unit 20 as will be described below, and is transmitted to the grasping unit 40 through the sheath 30. It is to be noted that, although not shown in FIG. 1, as is publicly known, a member may be interposed between the handle unit 11 and the handle unit 12 so as to apply some load to the rotating operation force of an operator to stably perform rotating operation.

The sheath 30 has a tubular shape, for example, a cylindrical body with a diameter of several mm and a length of about ten or so cm. The sheath 30 is fixed to the front end unit of the operation unit 10 so as to be parallel or concentric with a sliding space in which the transmission mechanism unit 20 is internally included.

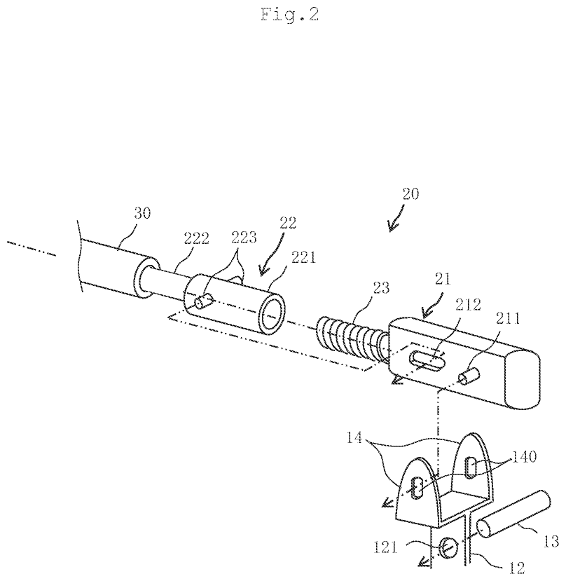

FIG. 2 is an assembly exploded view near the operation unit 10 of the grasping forceps 1. The transmission mechanism unit 20 is furnished inside the housing unit 10a of the operation unit 10, and a first movable unit 21, a second movable unit 22, and a spring 23 are arranged in the front-rear direction. The first movable unit 21, the second movable unit 22, and the spring 23 are slidable in the front-rear direction in the sliding space formed in the housing unit 10a.

The first movable unit 21 is a rectangular cylinder with a long cross section in an up-down direction and a bottom on the rear end side (the right side of FIG. 2). The first movable unit 21 has a pin 211 disposed perpendicularly at symmetrical positions on both left and right sides, and a long hole 212 long in the front-rear direction is bored near the front end of the pin 211.

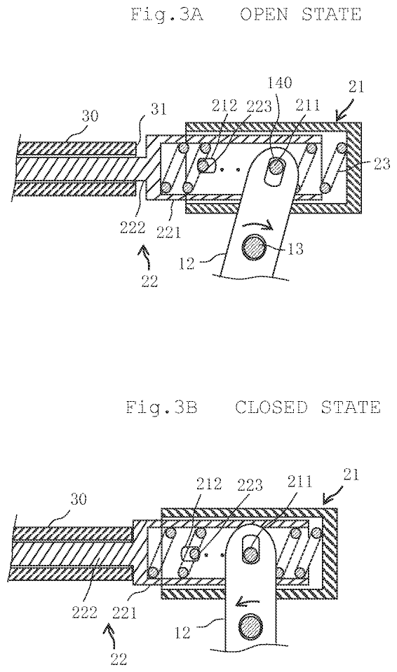

The second movable unit 22 has a cylindrical body 221 in the present embodiment and is configured to have a size enough to be loosely fitted (see FIG. 3A and FIG. 3B) into the first movable unit 21. At the front end of the cylindrical body 221, a long shaft 222 with a predetermined diameter is extended concentrically. In addition, a pin 223 is disposed perpendicularly at symmetrical positions on both left and right sides of the cylindrical body 221. The pin 223 is fitted into the long hole 212 of the first movable unit 21. The fitting of the pin 223 in the long hole 212 may be performed, for example, by inserting the cylindrical body 221 into the first movable unit 21 with the pin 223 located on the upper and lower sides and then rotating 90 degrees around the axis. The spring 23 functions as a biasing member and is loosely fitted into the cylindrical body 221 by a coil spring, for example (see FIG. 3A and FIG. 3B). The shaft 222 is loosely fitted into the sheath 30.

In addition, at the upper end of the handle unit 12 of the operation unit 10, a pair of left and right support plates 14 are disposed perpendicularly, and a long hole 140 long in the up-down direction is bored in a side surface of both the support plates 14. The pin 211 of the first movable unit 21 is fitted into the long hole 140. The pin 13 is internally fitted into a circle hole 121 bored in the handle unit 12. Next, an operation of the transmission mechanism unit 20 according to the rotating operation of the handle unit 12 will be described with reference to FIG. 3A and FIG. 3B.

FIG. 3A and FIG. 3B are side cross-sectional [symbol] views illustrating a motion of the operation unit 10 of the grasping forceps 1, FIG. 3A shows an open state of the grasping unit 40, and FIG. 3B shows a closed state of the grasping unit 40.

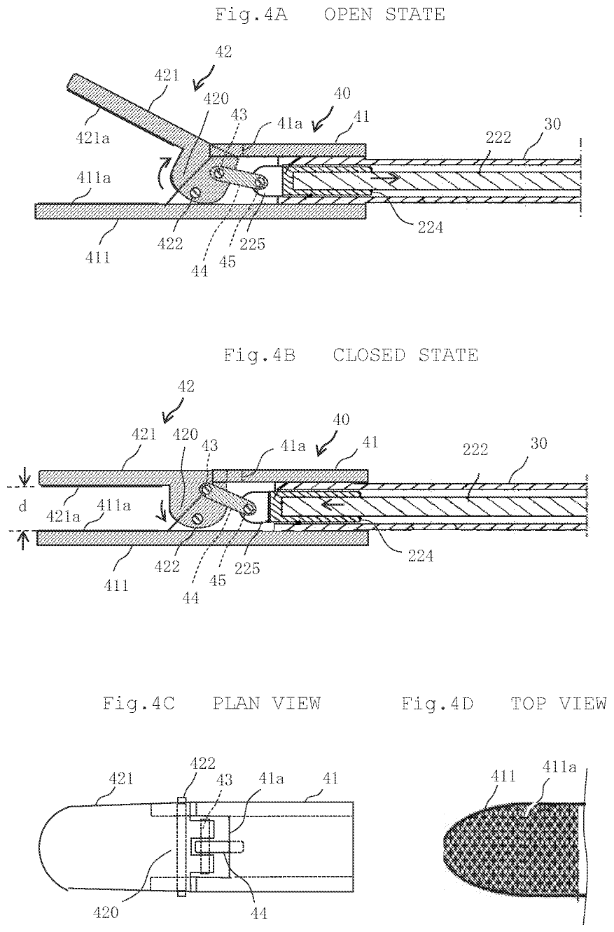

FIG. 3A shows a state in which the handle unit 12 grasped by the operator rotates clockwise around the pin 13 as shown by an arrow. At this time, the rotating force of the handle unit 12 is transmitted to the first movable unit 21 through the pin 211, and the first movable unit 21 slides backward (the right side of FIG. 3A and FIG. 3B). In addition, the long hole 212 presses the pin 223 backward, so that the second movable unit 22 also slides backward. Therefore, the front end of the cylindrical body 221 is separated from a part being a rear end 31 of the sheath 30 and functioning as a stopper, against biasing force of the spring 23. The backward sliding of the cylindrical body 221 causes the shaft 222 to slide backward. Then, the backward sliding of the shaft 222, as will be described in FIG. 4A, causes the front end of the grasping unit 40 to be in the open state.

FIG. 3B shows a state in which the handle unit 12 grasped by the operator rotates counterclockwise as shown by an arrow. At this time, the rotating force of the handle unit 12 is transmitted to the first movable unit 21 through the pin 211, and the first movable unit 21 slides forward. The long hole 212 presses the pin 223 frontward from the middle of this slide, so that the second movable unit 22 also slides forward. Therefore, the front end of the cylindrical body 221 also receives the biasing force of the spring 23, and slides into contact with the rear end 31 of the sheath 30. That is to say, the shaft 222 is slid in the sheath 30 only by a predetermined size in the direction of the front end, which, as will be described in FIG. 4B, causes the front end of the grasping unit 40 to be in the closed state.

FIG. 4A to FIG. 4D are views illustrating the motion of the grasping unit 40 of the grasping forceps 1, FIG. 4A is a side cross-sectional view in the open state, FIG. 4B is a side cross-sectional view in the closed state, FIG. 4C is a plan view of the grasping unit 40, and FIG. 4D is a top view showing an example of machining of a grasping surface of a lower jaw unit.

As shown in FIG. 4A to FIG. 4D, the grasping unit 40 includes a lower grasping unit 41, an upper grasping unit 42, pins 43 and 45, and a link 44. The pins 43 and 45 and the rod-shaped link 44 function as an opening and closing mechanism (a link unit). In addition, a metal fitting 224 and a pin support plate 225 are attached to the front end of the shaft 222. The pin support plate 225 axially supports the pin 45 in a left-right direction.

The lower grasping unit 41 is cylindrically shaped at a base end, and is externally attached to the front end of the sheath 30. The lower grasping unit 41 is mainly configured of a cylindrical unit near the base end, and a lower side part near the front end in a circumferential direction is machined flat and extended by a predetermined length to be formed as a lower jaw unit 411. The grasping surface 411a of the lower jaw unit 411, as shown in FIG. 4D, is a machined surface that has frictional resistance to increase graspability, and is diamond machined, for example.

The upper grasping unit 42 includes a rotating unit 420 and an upper jaw unit 421. The upper jaw unit 421 is extended from a front upper unit of the rotating unit 420 with a width, thickness, and length corresponding to the lower jaw unit 411. The grasping surface 421a of the upper jaw unit 421 is machined in the same way as the grasping surface 411a.

The upper jaw unit 421 is axially supported by the axis 422 so as to be relatively rotatable to the lower jaw unit 411. More specifically, the rotating unit 420, as shown in FIG. 4C, is internally fitted into a through hole in the left-right direction (the up-down direction of FIG. 4C) and is rotatably supported by the axis 422 axially supported near a lateral side of the lower grasping unit 41.

Furthermore, the link unit is interposed between the rotating unit 420 and the pin support plate 225. The link unit is configured by a pin 43 disposed over and axially supported by a pair of projection units formed across a gap on the rear end surface of the rotating unit 420 as shown in FIG. 4C, a pin 45 axially supported by the pin support plate 225 in the left-right direction, and a plate-shaped link 44 of which both ends are axially supported and coupled by the pins 43 and 45.

As shown in FIG. 4A, in a state in which the shaft 222 is slid backward (the right side of FIG. 4A to FIG. 4D), by moving the pin 45 backward and pulling the pin 43 backward through the link 44, the rotating unit 420 rotates clockwise around the axis 422 and opens the upper jaw unit 421.

In addition, as shown in FIG. 4B, in a state in which the shaft 222 is slid forward, by moving the pin 45 forward and pushing the pin 43 forward through the link 44, the rotating unit 420 rotates counterclockwise around the axis 422 and closes the upper jaw unit 421. In the present embodiment, the upper jaw unit 421 in the closed state is set parallel to the grasping surface the lower jaw unit 411. It is to be noted that an angle and a gap dimension d (see FIG. 4B) of the upper jaw unit 421 in the closed state are designed based on the size of the rotating unit 420, the position of the pins 43 and 45 to the axis 422, the length of the link 44, and also the shape of the first and second movable units 21 and 22 of the transmission mechanism unit 20. A notch 41a shown in FIG. 4A to FIG. 4C prevents interference with the rotating unit 420.

In the present embodiment, in the state of FIG. 4B, when the operator further tightly grasps the handle unit 12, greater stress may act on the pins 211 and 223 as well as the pins 43 and 45, which may damage the grasping forceps 1. For such a case, the pins 43 and 45 at a part to be interpolated in the body are set so as not to be easily deformed or broken as compared with the pin 211. More specifically, the pins 43 and 45 are designed to have a large diameter or adopt a strong material as compared with the other pin 211, which makes it possible to set higher bending strength.

The gap dimension d is prepared in a plurality of kinds of sizes according to the thickness, softness (flexibility), or the like of a grasping target. The grasping target includes a surgical cell sheet made of collagen or the like, meniscus, cartilage, a regenerative medical material, a cardiac muscle sheet, an intestinal membrane, various internal membranes, and other medical devices and materials, in addition to living tissue. In addition, it is applicable to grasping forceps for treatment under not only endoscopic vision but also direct vision. The parallel and necessary gap between the upper and lower jaw units 411 and 421, when tissue or the like to be grasped is grasped, prevents the tissue from being damaged or destroyed. The gap is set to a dimension such that a deformation ratio of the thickness of the tissue to be grasped may be 20% or less or preferably 10% or less. For example, when the target is flexible or fragile tissue and has a thickness of 3 mm, it is preferable to provide a gap such that the gap dimension d=2.5 mm. In this way, as the grasping forceps 1, the gap dimension d=3 mm, 5 mm, and 7 mm, for example, are applicable according to the grasping target.

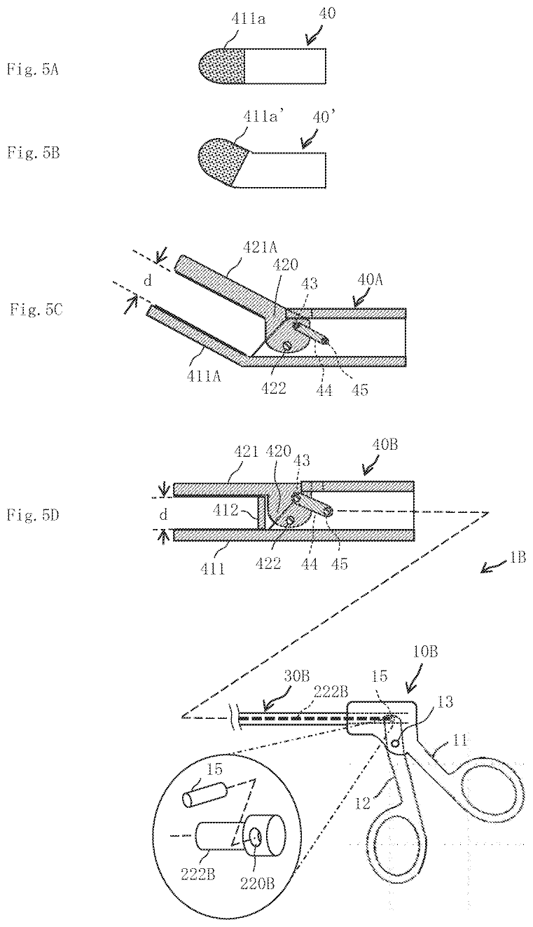

FIG. 5A to FIG. 5D are views showing various shapes of the grasping unit, FIG. 5A is a plan view showing a shape in which upper and lower jaw units extend straight, according to the present embodiment, FIG. 5B is a plan view showing a shape in which the upper and lower jaw units bend to one of the left and right sides, for example, to the right, FIG. 5C is a side cross-sectional view showing a shape in which the upper and lower jaw units bend to one of the upper and lower directions, for example, to the upper side, and FIG. 5D is a side cross-sectional view showing a shape in which a stopper is disposed perpendicularly to one of the facing surfaces of the upper and lower jaw units, for example, the grasping surface of the lower jaw unit.

In a procedure, in addition to a case in which the tissue or the like that is grasped by the grasping forceps is taken in and out against an affected part of a living body, a case in which the affected part is on the back side of the living body and the tissue is not easily taken in and out directly may be considered. The grasping forcipes 40′ and 40A shown in FIG. 5B and FIG. 5C have a shape in which a grasping surface 411a′ is bent in the left-right direction with 30°, for example, or the lower jaw unit 411A and the upper jaw unit 421A are bent in either the up or down direction with 20°, for example. The adoption of the grasping forceps of such a bent shape facilitates the operation to take the tissue in and out of the affected part regardless of the location of the affected part.

In addition, although the present embodiment shows a mode with the rear end 31 (the stopper) of the sheath 30 provided near the operation unit 10, the present invention is also applicable to a mode without a stopper provided near the operation unit 10. In FIG. 5D, near an operation unit 10B in grasping forceps 1B, at the rear end of a shaft 222B internally fitted into a sheath 30B, a vertically long hole 220B extending in the left-right direction as shown in the partially enlarged drawing is bored, and a pin 15 connected to the handle unit 12 is loosely fitted into this long hole 220B. In addition, a projecting member 412 is disposed perpendicularly to the grasping surface of the lower jaw unit 411, near a grasping unit 40B. The projecting member 412 may be a plate-shaped body or a rod-shaped body, for example. The height dimension of the projecting member 412, when the upper jaw unit 421 is in the closed state, is set so that the grasping surfaces with the lower jaw unit 411 may be parallel to each other. In this way, the closed state is first regulated at an angle at which the grasping surfaces are parallel to each other, so that the object to be grasped is able to be accurately, reliably, and stably grasped. In addition, in this case, the pins 43 and 45 have higher strength than the pin 15, so that breakage and destruction of the pins 43 and 45 near the grasping unit 40B against overtightening of the handle unit 12, as well as the consequent dropping of a component are prevented.

In addition, a limiter is placed near the operation unit 10, and a configuration to regulate, after the grasping unit 40 is in the closed state, the transmission of the rotating operation force toward the grasping unit 40 due to further tightly grasping of the handle unit 12 may be adopted.

As described above, the grasping forceps according to the present invention preferably include a tubular sheath, a shaft furnished inside the sheath, an operation unit provided near a base end of the sheath and sliding the shaft by converting rotating operation force to a handle into an axial direction of the shaft through a first pin, and a grasping unit provided near a front end of the sheath and enabling upper and lower jaw units to be open and closed, and the grasping unit preferably includes a link unit that couples the upper and lower jaw units and the front end of the shaft through a second pin and opens and closes the upper and lower jaw units according to a slide of the shaft, and the link unit, when the upper and lower jaw units are in a closed state, preferably sets facing grasping surfaces to be parallel and have a predetermined gap.

According to the present invention, when the upper and lower jaw units are in the closed state, the link unit is configured so that the facing grasping surfaces may be parallel and have a predetermined gap, so that various objects to be grasped are able to be accurately, reliably, and stably grasped without being damaged, broken, or dropped.

In addition, the operation unit preferably includes a regulation unit that, in a case in which the upper and lower jaw units are in the closed state and further the rotating operation force exceeds a predetermined force, regulates the transmission of the rotating operation force to the shaft. According to this configuration, even when a handle unit is overtightened, the transmission to the shaft is regulated, which prevents a structural component being an insertion unit into a body from being damaged, destroyed, and consequently dropped.

In addition, the regulation unit is preferably the first pin, and the first pin preferably has a lower strength than the second pin. Moreover, the first pin has a smaller diameter than the second pin. According to such a configuration, the regulation unit is achieved with a simple configuration.

In addition, the link unit preferably openably and closably swings one of the upper and lower jaw units to the other. According to this configuration, the one of the upper and lower jaw units is configured to be movable, which provides forceps with a simple configuration and high operability.

In addition, the upper and lower jaw units are preferably bent by a predetermined angle in the axial direction of the shaft. According to this configuration, even when an affected part is on the back side of the living body, an object to be grasped is easily taken in and out.

### REFERENCE SIGNS LIST

1 grasping forceps
10 operation unit
11, 12 handle unit
20 transmission mechanism unit
21 first movable unit
211, 15 pin (first pin, regulation unit)
22 second movable unit
222 shaft
30 sheath
40 grasping unit
41 lower grasping unit
42 upper grasping unit
411 lower jaw unit
421 upper jaw unit
43, 45 pin (link unit, second pin)
44 link (link unit)

1. Grasping forceps comprising:
a cylindrical sheath;
a shaft furnished inside the sheath;
an operation unit provided near a base end of the sheath and sliding the shaft by converting rotating operation force to a handle into an axial direction of the shaft through a first pin; and
a grasping unit provided near a front end of the sheath and enabling upper and lower jaw units to be open and closed, wherein:
the grasping unit includes a link unit that couples the upper and lower jaw units and the front end of the shaft through a second pin and opens and closes the upper and lower jaw units according to a slide of the shaft; and
the link unit, when the upper and lower jaw units are in a closed state, sets facing grasping surfaces to be parallel and have a predetermined gap.

2. The grasping forceps according to claim 1, wherein the operation unit includes a regulation unit that, in a case in which the upper and lower jaw units are in the closed state and the rotating operation force exceeds a predetermined force, regulates transmission of the rotating operation force to the shaft.

3. The grasping forceps according to claim 2, wherein:
the regulation unit is the first pin; and
the first pin has a lower strength than the second pin.

4. The grasping forceps according to claim 3, wherein the first pin has a smaller diameter than the second pin.

5. The grasping forceps according to claim 1, wherein the link unit openably and closably swings one of the upper and lower jaw units to another.

6. The grasping forceps according to claim 1, wherein the upper and lower brim jaw units are bent by a predetermined angle in the axial direction of the shaft.
